SEQUENCE LISTING (11 sequences)

SEQ ID NO: 1 - DNA, 2433 bp, Rattus norvegicus
  CDS (262)..(2238)
gaattcggcacgagcaatttcctcatctgatttctaaaggaagctacttgcgatggtcctctgcaacctcgtgtgtcgattgctaagtcattgctactcgcattggaatgatctctaccccgcaatggagagtggcatgccatcagaaagaaaaacgaacggacaaagagctcagtagaaactctggcagcgagaacacagagaaacgcatggagatgagctaagtcgctgagcggtgggctgacagtgtaccggttcaggatgttgcacgtgaacgacttgcctcctcccaggcgacactcgtggatatgctttgatgtggaaaatggcccttctccaggtcggagcccactggaccctcaagccagctcttcttcaggactggtacttcatgccgccttccctgggcacagccaacgcagagagtcttttctctacagatccgacagcgactatgacttgtcaccaaaagcgatgtcaaggaactcctcacttcccagcgaacaacacggcgatgacctgattgtcactccttttgcccaggttcttgccagcttgcgaagcgtaagaaacaatttcaccctgctgacaaaccttcacggagcaccgaacaagaggtcgccagcggctagtcaggctccagtcaccagagtcagcctgcaagaagaatcatatcagaaactagcaatggagacgctggaggaactagactggtgcctagaccagctagagaccatccagacctaccgctctgtcagcgagatggcttcaaacaagttcaaaaggatgctgaaccgggagctgacacacctctcagagatgagcagatcagggaaccaagtgtctgaatacatttcgaacacgttcttagacaagcagaacgatgtggaaatcccatctcccacccagaaggacagggagaagaagaagaagcagcagctcatgacccagataagtggagtgaagaaactgatgcacagctcaagcctgaacaacacaagcatctcacgctttggagtcaacacggaaaatgaggatcatctagccaaggagctggaagacctgaacaaatggggccttaacatcttcaacgtggctgggtactcccataatcggcccctcacatgcatcatgtacgccattttccaggaaagagaccttctaaagacgtttaaaatctcctccgacaccttcgtaacctacatgatgactttagaagaccattaccattctgatgtggcgtatcacaacagcctgcacgctgctgacgtggcccagtcaacgcacgttctcctctctacgccagcactggatgctgtcttcacagacctggaaatcctggctgccatttttgcagctgccatccatgatgttgatcatcctggagtctccaatcagtttctcatcaatacaaattccgaacttgctttgatgtataatgacgaatctgtgctggaaaaccatcacctcgctgtgggattcaagctccttcaagaggaacattgcgacatctttcagaatcttaccaagaagcaacgccagacactcaggaaaatggtgattgacatggtgttagcaactgatatgtccaagcacatgagcctcctggctgaccttaaaacgatggtagaaaccaaaaaggtgacgagctccggtgttctcctcctggacaactatactgaccggatacaggttcttcgcaacatggtacattgtgcagacctgagcaaccctaccaagtccttggagttgtatcggcaatggactgatcgcatcatggaggagtttttccaacagggagacaaagaacgggagaggggaatggagattagcccaatgtgtgataaacacacagcttctgtggaaaagtcccaggttggtttcattgactacattgtccatccattgtgggagacctgggcagacctggttcagcctgatgctcaagacattttggacacactagaagataacaggaactggtaccagagtatgattccccagagcccctctccaccactggacgagaggagcagggactgccaaggccttatggagaagtttcagttcgaactgacccttgaagaagaggattctgaaggaccggaaaaggagggagaaggccccaactatttcagcagcacaaagacactttgtgtgatcgatccagagaacagggattctctggaagagactgacatagacattgccacagaagacaagtctctgatcgacacataatctccctctgtgtggaggtgaacattctatccttgacgagcatgccagctgagtggtagggcccacctaccagagccaaggcctgcacaaaacaaaggccacctggctttgcagttacttgagtttggagccagaatgcaaggccgtgaagcaaatagcagttccgtgctgccttgccttgccggcgagctt

SEQ ID NO: 2 - protein, 659 aa, Rattus norvegicus
MLHVNDLPPPRRHSWICFDVENGPSPGRSPLDPQASSSSGLVLHAAFPGHSQRRESFLYRSDSDYDLSPKAMSRNSSLPSEQHGDDLIVTPFAQVLASLRSVRNNFTLLTNLHGAPNKRSPAASQAPVTRVSLQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFKISSDTFVTYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFQNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDERSRDCQGLMEKFQFELTLEEEDSEGPEKEGEGPNYFSSTKTLCVIDPENRDSLEETDIDIATEDKSLIDT

SEQ ID NO: 3 - DNA, 51 bp, Rattus norvegicus
  CDS (1)..(51)
atgttgcacgtgaacgacttgcctcctcccaggcgacactcgtggatatgc

SEQ ID NO: 4 - protein, 17 aa, Rattus norvegicus
MLHVNDLPPPRRHSWIC

SEQ ID NO: 5 - DNA, 3022 bp, Rattus norvegicus
  CDS (325)..(2532)
gaattcggcacgaggcgcgggggcgggggcggcgtgtagtggcagacggccgcagggattatgaatgggggtgggggccggcgagttgaggttccacccgggatcgtccgcaccggctgatgggcacgcagggctgcgtgtaatcctccagcctcggtggagggaggctgcagcgagcgccggctggcagtaagggttcttctgcaaaagtcccctgcggttgcgcgcgtggagtgccggggagctcggccaggtctagtctgcagtcagcaaagctgcagcaaacagcagacatctccagaggagctgtttgccacatctataatgaagaaaagtaggagtgtgatggccgtgactgcagatgataatcttaaggactattttgaatgtagcttgagtaaatcctacagttcttccagttatacccttgggattgacctctggagaggcagaaggtgctgttcaggaaacttacagttgccaccattgtcccagagacaaagtgaaagggcaaggacacctgagggagatggcatttccaggccaaccacgctacctttgacgacacttcccagcattgctataacaactgtaagccaggagtgctttgatgtggaaaatggcccttctccaggtcggagcccactggaccctcaagccagctcttcttcaggactggtacttcatgccgccttccctgggcacagccaacgcagagagtcttttctctacagatccgacagcgactatgacttgtcaccaaaagcgatgtcaaggaactcctcacttcccagcgaacaacacggcgatgacctgattgtcactccttttgcccaggttcttgccagcttgcgaagcgtaagaaacaatttcaccctgctgacaaaccttcacggagcaccgaacaagaggtcgccagcggctagtcaggctccagtcaccagagtcagcctgcaagaagaatcatatcagaaactagcaatggagacgctggaggaactagactggtgcctagaccagctagagaccatccagacctaccgctctgtcagcgagatggcttcaaacaagttcaaaaggatgctgaaccgggagctgacacacctctcagagatgagcagatcagggaaccaagtgtctgaatacatttcgaacacgttcttagacaagcagaacgatgtggaaatcccatctcccacccagaaggacagggagaagaagaagaagcagcagctcatgacccagataagtggagtgaagaaactgatgcacagctcaagcctgaacaacacaagcatctcacgctttggagtcaacacggaaaatgaggatcatctagccaaggagctggaagacctgaacaaatggggccttaacatcttcaacgtggctgggtactcccataatcggcccctcacatgcatcatgtacgccattttccaggaaagagaccttctaaagacgtttaaaatctcctccgacaccttcgtaacctacatgatgactttagaagaccattaccattctgatgtggcgtatcacaacagcctgcacgctgctgacgtggcccagtcaacgcacgttctcctctctacgccagcactggatgctgtcttcacagacctggaaatcctggctgccatttttgcagctgccatccatgatgttgatcatcctggagtctccaatcagtttctcatcaatacaaattccgaacttgctttgatgtataatgacgaatctgtgctggaaaaccatcacctcgctgtgggattcaagctccttcaagaggaacattgcgacatctttcagaatcttaccaagaagcaacgccagacactcaggaaaatggtgattgacatggtgttagcaactgatatgtccaagcacatgagcctcctggctgaccttaaaacgatggtagaaaccaaaaaggtgacgagctccggtgttctcctcctggacaactatactgaccggatacaggttcttcgcaacatggtacattgtgcagacctgagcaaccctaccaagtccttggagttgtatcggcaatggactgatcgcatcatggaggagtttttccaacagggagacaaagaacgggagaggggaatggagattagcccaatgtgtgataaacacacagcttctgtggaaaagtcccaggttggtttcattgactacattgtccatccattgtgggagacctgggcagacctggttcagcctgatgctcaagacattttggacacactagaagataacaggaactggtaccagagtatgattccccagagcccctctccaccactggacgagaggagcagggactgccaaggccttatggagaagtttcagttcgaactgacccttgaagaagaggattctgaaggaccggaaaaggagggagaaggccccaactatttcagcagcacaaagacactttgtgtgatcgatccagagaacagggattctctggaagagactgacatagacattgccacagaagacaagtctctgatcgacacataatctccctctgtgtggaggtgaacattctatccttgacgagcatgccagctgagtggtagggcccacctaccagagccaaggcctgcacaaaacaaaggccacctggcctttgcagttacttgagtttggagccagaatgcaaggccgtgaagcaaatagcagttccgtgctgccttgccttgccggcgagcttggcggagacccgcagctgtagtagaagccagttcccagcacagctaaatggcttgaaaacagaggacagaaagctgagagattgctctgcaataggtgttgaggggctgtcccgacaggtgactgaactcactaacaacttcatctataaatctcacccatcctgttgtctgccaacctgtgtgccttttttgtaaaatgttttcgtgtctttgaaatgcctgttgaatatctagagtttagtacctccttctacaaacttttttgagtctttctgggaaaaaaaaacctgcag

SEQ ID NO: 6 - protein, 736 aa, Rattus norvegicus
MKKSRSVMAVTADDNLKDYFECSLSKSYSSSSYTLGIDLWRGRRCCSGNLQLPPLSQRQSERARTPEGDGISRPTTLPLTTLPSIAITTVSQECFDVENGPSPGRSPLDPQASSSSGLVLHAAFPGHSQRRESFLYRSDSDYDLSPKAMSRNSSLPSEQHGDDLIVTPFAQVLASLRSVRNNFTLLTNLHGAPNKRSPAASQAPVTRVSLQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFKISSDTFVTYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFQNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDERSRDCQGLMEKFQFELTLEEEDSEGPEKEGEGPNYFSSTKTLCVIDPENRDSLEETDIDIATEDKSLIDT

SEQ ID NO: 7 - protein, 736 aa, Homo sapiens; identical to SEQ ID NO: 6

SEQ ID NO: 8 - protein, 564 aa, Rattus norvegicus
MKEQGGTVSGAGSSRGGGDSAMASLQPLQPNYLSVCLFAEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFKISSDTFVTYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFQNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDERSRDCQGLMEKFQFELTLEEEDSEGPEKEGEGPNYFSSTKTLCVIDPENRDSLEETDIDIATEDKSLIDT

SEQ ID NO: 9 - protein, 721 aa, Rattus norvegicus
MTAKNSSKELPASESEVCIKTFKEQMRLELELPKLPGNRPTSPKISPRSSPRNSPCFFRKLLVNKSIRQRRRFTVAHTCFDVENGPSPGRSPLDPQASSSSGLVLHAAFPGHSQRRESFLYRSDSDYDLSPKAMSRNSSLPSEQHGDDLIVTPFAQVLASLRIVRNNFTLLTNLHGAPNKRSPAASQAPVTRVSLQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFKISSDTFVTYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFQNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDERSRDCQGLMEKFQFELTLEEEDSEGPEKEGEGPNYFSSTKTLCVIDPENRDSLEETDIDIATEDKSLIDT

SEQ ID NO: 10 - DNA, 2647 bp, Rattus norvegicus
gtcttgtcatcaggagacctcattttacctctaggttaagggagagaatctatgaagagaaaggaatagtctgtgtctcggtcttgtccgggtcagtgtttctgagagctcacagtggccacctgaagcatttttccccagaatgaatgactgccctgcctgagaacagaagagccaaacagttccccccacatggccatagggagctggtttcatttagaagaaaagcaaagagaggggaaagcctccctcatttctcctccggacggcaaacattcagaaatgacatcacacaccccacagccccgggatgactaaggcagaagtagcctgagaaaactctgctctgccctgagttttagggcacagttatgcagatgagcgtctgggcgcaggttcccgccttcttcctctgaggaagtttcttggtagatcactgacacctcatcccggcgagggggtgaaaacttggcaccagccactccccctcccgggcagagcaccagaaagagcttggaagcaaggagtcggcaagcaaacaatgaaggagcaagggggcaccgtcagtggcgccgggagcagccgaggcggaggagactcggctatggccagcctgcagccgctgcagcctaactacctgtctgtgtgtttgtttgcagaagaatcatatcagaaactagcaatggagacgctggaggaactagactggtgcctagaccagctagagaccatccagacctaccgctctgtcagcgagatggcttcaaacaagttcaaaaggatgctgaaccgggagctgacacacctctcagagatgagcagatcagggaaccaagtgtctgaatacatttcgaacacgttcttagacaagcagaacgatgtggaaatcccatctcccacccagaaggacagggagaagaagaagaagcagcagctcatgacccagataagtggagtgaagaaactgatgcacagctcaagcctgaacaacacaagcatctcacgctttggagtcaacacggaaaatgaggatcatctagccaaggagctggaagacctgaacaaatggggccttaacatcttcaacgtggctgggtactcccataatcggcccctcacatgcatcatgtacgccattttccaggaaagagaccttctaaagacgtttaaaatctcctccgacaccttcgtaacctacatgatgactttagaagaccattaccattctgatgtggcgtatcacaacagcctgcacgctgctgacgtggcccagtcaacgcacgttctcctctctacgccagcactggatgctgtcttcacagacctggaaatcctggctgccatttttgcagctgccatccatgatgttgatcatcctggagtctccaatcagtttctcatcaatacaaattccgaacttgctttgatgtataatgacgaatctgtgctggaaaaccatcacctcgctgtgggattcaagctccttcaagaggaacattgcgacatctttcagaatcttaccaagaagcaacgccagacactcaggaaaatggtgattgacatggtgttagcaactgatatgtccaagcacatgagcctcctggctgaccttaaaacgatggtagaaaccaaaaaggtgacgagctccggtgttctcctcctggacaactatactgaccggatacaggttcttcgcaacatggtacattgtgcagacctgagcaaccctaccaagtccttggagttgtatcggcaatggactgatcgcatcatggaggagtttttccaacagggagacaaagaacgggagaggggaatggagattagcccaatgtgtgataaacacacagcttctgtggaaaagtcccaggttggtttcattgactacattgtccatccattgtgggagacctgggcagacctggttcagcctgatgctcaagacattttggacacactagaagataacaggaactggtaccagagtatgattccccagagcccctctccaccactggacgagaggagcagggactgccaaggccttatggagaagtttcagttcgaactgacccttgaagaagaggattctgaaggaccggaaaaggagggagaaggccccaactatttcagcagcacaaagacactttgtgtgatcgatccagagaacagggattctctggaagagactgacatagacattgccacagaagacaagtctctgatcgacacataatctccctctgtgtggaggtgaacattctatccttgacgagcatgccagctgagtggtagggcccacctaccagagccaaggcctgcacaaaacaaaggccacctggctttgcagttacttgagtttggagccagaatgcaaggccgtgaagcaaatagcagttccgtgctgccttgccttgccggcgagcttggcgagacccgcagctgtagtagaagccagttcccagcacagctaaatggcttgaaaacagaggacagaaagctgagagattgctctgcaataggtgttgaggggctgtcccgacaggtgactgaactcactaacaacttcatctataaatctcacccatcctgttgtctgccaacctgtgtgccttttttgtaaaatgttttcgtgtctttgaaatgc

SEQ ID NO: 11 - DNA, 3133 bp, Rattus norvegicus
gaattcgcgcacgagagcacatgctggatggactctggttccgccctttgtgcagacaaaagtgactgggtggccaggctttgcttactgtctgagttaatgaagcttgtttgataaggttttctttcaaaaaaaaattacatataaaggatttatcaaaagccctcatgaatatttcatgagttgatacattcggctgaatggattcagtgagtctcagtgtgtaacttgcacacaagccccatccacaaggaggctggtgacagaggaagcactttggcgcattttcagaggcaaaggcagcctgataaagctcctgtgacaggctgacttgccattctcccagtatgctgctcttgctctgaagtgctccaggattggaaccatcacggcttcccaaattagcctaggacgagtgtgcggacccagcagccttttaacctgcggcagtgcctttgctatgttcaagactgttgttgtggatggtgaaagctagcgcgccacacgagacatgacagcaaaaaattcttccaaagaacttccagcttctgaatctgaggtttgcataaagactttcaaggagcagatgcgcttggaacttgagcttccaaagctaccaggaaacagacctacatctcccaaaatttctccacgcagttcaccaaggaattcaccatgctttttcagaaagttgctggtgaataaaagcatccgacagcggcgtcgcttcactgtggctcatacatgctttgatgtggaaaatggcccttctccaggtcggagcccactggaccctcaagccagctcttcttcaggactggtacttcatgccgccttccctgggcacagccaacgcagagagtcttttctctacagatccgacagcgactatgacttgtcaccaaaagcgatgtcaaggaactcctcacttcccagcgaacaacacggcgatgacctgattgtcactccttttgcccaggttcttgccagcttgcgaatcgtaagaaacaatttcaccctgctgacaaaccttcacggagcaccgaacaagaggtcgccagcggctagtcaggctccagtcaccagagtcagcctgcaagaagaatcatatcagaaactagcaatggagacgctggaggaactagactggtgcctagaccagctagagaccatccagacctaccgctctgtcagcgagatggcttcaaacaagttcaaaaggatgctgaaccgggagctgacacacctctcagagatgagcagatcagggaaccaagtgtctgaatacatttcgaacacgttcttagacaagcagaacgatgtggaaatcccatctcccacccagaaggacagggagaagaagaagaagcagcagctcatgacccagataagtggagtgaagaaactgatgcacagctcaagcctgaacaacacaagcatctcacgctttggagtcaacacggaaaatgaggatcatctagccaaggagctggaagacctgaacaaatggggccttaacatcttcaacgtggctgggtactcccataatcggcccctcacatgcatcatgtacgccattttccaggaaagagaccttctaaagacgtttaaaatctcctccgacaccttcgtaacctacatgatgactttagaagaccattaccattctgatgtggcgtatcacaacagcctgcacgctgctgacgtggcccagtcaacgcacgttctcctctctacgccagcactggatgctgtcttcacagacctggaaatcctggctgccatttttgcagctgccatccatgatgttgatcatcctggagtctccaatcagtttctcatcaatacaaattccgaacttgctttgatgtataatgacgaatctgtgctggaaaaccatcacctcgctgtgggattcaagctccttcaagaggaacattgcgacatctttcagaatcttaccaagaagcaacgccagacactcaggaaaatggtgattgacatggtgttagcaactgatatgtccaagcacatgagcctcctggctgaccttaaaacgatggtagaaaccaaaaaggtgacgagctccggtgttctcctcctggacaactatactgaccggatacaggttcttcgcaacatggtacattgtgcagacctgagcaaccctaccaagtccttggagttgtatcggcaatggactgatcgcatcatggaggagtttttccaacagggagacaaagaacgggagaggggaatggagattagcccaatgtgtgataaacacacagcttctgtggaaaagtcccaggttggtttcattgactacattgtccatccattgtgggagacctgggcagacctggttcagcctgatgctcaagacattttggacacactagaagataacaggaactggtaccagagtatgattccccagagcccctctccaccactggacgagaggagcagggactgccaaggccttatggagaagtttcagttcgaactgacccttgaagaagaggattctgaaggaccggaaaaggagggagaaggccccaactatttcagcagcacaaagacactttgtgtgatcgatccagagaacagggattctctggaagagactgacatagacattgccacagaagacaagtctctgatcgacacataatctccctctgtgtggaggtgaacattctatccttgacgagcatgccagctgagtggtagggcccacctaccagagccaaggcctgcacaaaacaaaggccacctggctttgcagttacttgagtttggagccagaatgcaaggccgtgaagcaaatagcagttccgtgctgccttgccttgccggcgagcttggcgagacccgcagctgtagtagaagccagttcccagcacagctaaatggcttgaaaacagaggacagaaagctgagagattgctctgcaataggtgttgaggggctgtcccgacaggtgactgaactcactaacaacttcatctataaatctcacccatcctgttgtctgccaacctgtgtgccttttttgtaaaatgttttcgtgtctttgaaatgcctgttgaatatctagagtttagtacctccttctacaaactttttt

(19) United States
(12) Patent Application Publication
(10) Pub. No.: US 2003/0208056 A1
(43) Pub. Date: Nov. 6, 2003

(54) Two novel camp-specific phosphodiesterase (pde4b)isoforms and related technology

(76) Inventor: Graeme B. Bolger, Birmingham, AL (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/149,536

(22) PCT Filed: Dec. 14, 2000
(86) PCT No.: PCT/US00/34045

(51) Int. Cl. C07H 21/04; C12N 9/16; C12P 21/02; C12N 5/06; C07K 16/40

(52) U.S. Cl. 536/23.2; 435/196; 435/69.1; 435/320.1; 435/325; 530/388.26

(57) ABSTRACT

Two novel cAMP-specific isoforms of rat PDE4B are disclosed. pRPDE90 is a cDNA encoding a 659-amino acid-long protein with a large region corresponding to similar regions found in PDE4B1 and PDE4B3. It is separated from these isoforms by a 17-amino acid region found at its extreme amino-terminal end which shows no homology to any previously-cloned sequence. pRPDE89 is a rat cDNA which encodes a 726-amino acid-long protein which is 96% identical to the human PDE4B1 phosphodiesterase isoform.

### RELATED APPLICATIONS

This application is related to and claims the benefit of U.S. Provisional Application Serial No. 60/170,562 of Graeme B. Bolger, filed Dec. 14, 1999 and entitled “Two Novel cAMP-Specific Phosphodiesterase (PDE4B) Isoforms and Related Technology,” which is incorporated herein by reference.

### FIELD OF THE INVENTION

The present invention relates to cyclic AMP-specific phosphodiesterases (PDE4 enzymes), which help regulate physiological processes by hydrolizing cAMP, an intracellular signaling molecule. Specifically, the invention relates to two novel cAMP-specific phosphodiesterase isoforms which are expressed in many body tissues, including the brain.

### TECHNICAL BACKGROUND

Cyclic AMP (“cAMP”) is an intracellular signaling molecule involved in many important cellular processes. Specifically, cAMP is critical to signaling pathways which regulate physiological processes such as those involved in vascular smooth muscle, the immune system, and the brain. cAMP-specific phosphodiesterases, referred to as “PDE4 enzymes,” hydrolyze cAMP and thus regulate these pathways in cells. The cAMP-specific phosphodiesterases can be differentiated from other cyclic nucleotide phosphodiesterase (“PDE”) families by sequence homology in the catalytic region of the proteins as well as by their ability to be inhibited by a specific class of drugs, such as rolipram. Beavo, Physiol. Rev. 75:725-48 (1995). Rolipram and other specific PDE4 inhibitors have anti-depressant, anti-inflammatory and smooth muscle relaxant properties in humans. Houslay et al., Advances in Pharmacology, 44:225-342 (1998). PDE4 enzymes are also characterized by the presence of unique “signature” regions of sequence, called upstream conserved regions, or “UCR,” such as UCR1 and UCR2, which are located in the amino-terminal third of the proteins. Houslay, et al., supra; Bolger et al., Mol. Cell Biol., 13:6558-71 (1993).

PDE4 enzymes are also the closest vertebrate homologs of the dunce gene of Drosophila melanogaster, which was isolated as a mutation affecting learning and memory. Davis, Physiol. Rev. 76:299-317 (1996). The mammalian PDE4s are encoded by four genes (PDE4A, PDE4B, PDE4C and PDE4D), and it has been shown by several researchers that additional diversity in this family is produced by alternative mRNA splicing. Bolger et al., supra; Sette et al., J. Biol. Chem., 269(32):20806 (Aug. 12, 1994); Bolger et al., J. Biol. Chem., 271:1065-71 (1996); Bolger et al., Biochem. J., 328:539-48 (1997); Naro et al., Endocrinology, 137:2464-72 (1996); Huston et al., Biochem J. 328:549-56 (1997). See Houslay et al., supra, for a review of these findings. Often the alternatively-spliced isoforms have different tissue expression patterns, a fact which suggests that each may have a distinct function.

Intracellular signaling molecules are important since they transmit a signal received outside of the cell to target molecules in the cytosol, thus allowing a cell to react to changes in its environment. This transmission is generally a multistep process often having at least the general steps laid out in the following cAMP-specific sequence: First, an extracellular ligand binds to a plasma membrane-bound receptor molecule which has a binding domain extending into the extracellular space and a domain extending into the cytosol. Second, the binding of the ligand to the extracellular domain changes the conformation of the cytosolic domain, thus causing it to bind to a G-protein. Third, the G-protein, in turn, activates a plasma membrane enzyme which produces cAMP (adenylyl cyclase). Fourth, the cAMP then binds to target molecules in the cytosol, thus altering their conformation and activity. Finally, cyclic AMP-specific phosphodiesterases rapidly break down the cAMP, hydrolizing it to form adenosine 5′-monophosphate.

As seen in the final step, in order to use cAMP as a signaling molecule, a cell must be able to quickly manipulate the levels of cAMP present in response to signals transmitted to the outside of the cell. Cyclic AMP functions well in this respect, having been shown in some cases to respond to hormonal stimulation by increasing in cellular concentration by five-fold within seconds.

Such rapid changes in cAMP levels are possible due to a cell's ability to rapidly synthesize cAMP. Cells are also adapted to rapidly break down cAMP. Cyclic AMP is synthesized from ATP by adenylyl cyclase, an enzyme found in the plasma membrane of a cell. Cyclic AMP is hydrolized by cyclic AMP phosphodiesterases to form adenosine 5′-monophosphate (“5′-AMP”). These phosphodiesterases are found in many tissues, including specific regions of the brain.

It is known that certain cAMP-specific phosphodiesterases (“PDE4 enzymes”) are the targets of inhibitors. Some of these inhibitors have been shown to have positive effects on the brain, including exhibiting anti-depressant properties, memory-enhancing qualities, and other positive effects on the function of the central nervous system. Unfortunately, however, these beneficial effects are often accompanied by nausea and other gastrointestinal side effects. These negative side effects are likely mediated at least in part by the action of the inhibitors used on the brain. The number of isoforms of the PDE4 enzymes present in the brain is currently unknown, as is an understanding of which inhibitors affect which phosphodiesterases. Knowledge of novel isoforms of PDE4 enzymes would be a great advancement in the art, allowing researchers and health professionals to learn to target PDE4 inhibitors to specific isoforms and limit the effects of the inhibition to the desired, positive effects, while avoiding inhibition of those isoforms whose inhibition causes the deleterious side effects noted above.

From the foregoing, it will be appreciated that it would be an advancement in the art to identify additional PDE4 enzyme isoforms. Such identification would enable investigation of the patterns of isoform tissue expression, and thus allow selective targeting of specific isoforms with isoform-specific inhibitors, yielding effective use of the beneficial effects of inhibition while avoiding the deleterious ones.

Such novel PDE4 enzyme isoforms are disclosed herein.

### BRIEF SUMMARY OF THE INVENTION

The present invention relates to isoforms of cAMP-specific phosphodiesterase. Specifically, two rat cDNAs, pRPDE89 and pRPDE90, were isolated from a rat cerebral cortex cDNA library. Both of these were found to encode novel PDE4B isoforms. The invention thus comprises a first cDNA, pRPDE89, which encodes a protein identical in length to that encoded by the previously-described human PDE4B1 isoform known in the art. The protein encoded by both the rat and human genetic material is 736 amino acids in length. This rat cAMP-specific phosphodiesterase isoform is over 96% identical in sequence to the human PDE4B1 isoform.

The invention further comprises a second cDNA, pRPDE90, which encodes a novel protein of 659 amino acids, called PDE4B4. PDE4B4 has a novel N-terminal region of 17 amino acids which is not present in any other known PDE4B isoform. The remaining 642 amino acids of PDE4B4 are identical to those found in corresponding regions of the PDE4B1 and PDE4B3 isoforms. Without being bound to any particular theory, it is believed that the structures of the cDNAs encoding the PDE4B1, PDE4B3, and PDE4B4 isoforms are generated by alternative mRNA splicing and through the use of alternative promoters of the PDE4B gene. RNase protection and immunoblotting demonstrated the presence of mRNA and protein specific for each of the PDE4B1, PDE4B2, PDE4B3 and PDE4B4 isoforms, respectively, in a wide range of tissues, including various regions of the brain.

Since various inhibitors of cAMP phosphodiesterases have been shown to have anti-depressant and memory enhancement effects, the discovery of novel isoforms of PDE4B opens possibilities of better understanding and targeting such inhibitors to have more selective effects on the brain.

These and other features of the present invention will become apparent upon reference to the accompanying figures and upon reading the following detailed description and appended claims.

### BRIEF DESCRIPTION OF THE DRAWINGS

A more particular description of the invention briefly described above will be rendered by reference to the appended figures. These figures only provide information concerning typical embodiments of the invention and are not therefore to be considered limiting of its scope.

FIG. 1 shows the structure of mRNAs encoded by the rat PDE4B gene. The numbers 1-4 indicate transcripts represented by the following cDNAs: 1, PDE4B1 (pRPDE89 (SEQ ID NO: 5); GenBank™ AF202732); 2, PDE4B2 (pRPDE18 (SEQ ID NO: 8); GenBank™ L27058); 3, PDE4B3 (pRPDE74 (SEQ ID NO: 9); GenBank™ U95748); 4, PDE4B4 (pRPDE90 (SEQ ID NO: 1) and pRPDE92 (SEQ ID NO: 10); GenBank™ AF202733). The heavy bar indicates sequences homologous to other PDE4 isoforms, with the strongest regions of conservation (the catalytic region and UCR1 and UCR2) indicated by the cross-hatched areas. The thin, branched lines adjacent to the numbers indicate sequence regions unique to each isoform. The thin lines merge where the sequences of the various isoforms join the shared sequence. Small boxes indicate start codons and the asterisk indicates the common stop codon.

FIG. 2 shows an alignment of the amino acid sequences of human PDE4B1 (top, SEQ ID NO: 7) and rat PDE4B1 (bottom, SEQ ID NO: 6). The sequence of human PDE4B1 has been described previously (pTM72 in Bolger, Mol. Cell Biol. 13:6558-71 (1993), GenBank™ L20966). The sequence of PDE4B1 was deduced from the pRPDE89 cDNA.

FIG. 3 shows an alignment of the amino acid sequences of rat PDE4B1 (SEQ ID NO: 6), PDE4B2 (SEQ ID NO: 8), PDE4B3 (SEQ ID NO: 9), and PDE4B4 (SEQ ID NO: 2). The sequences are derived from the following cDNAs: PDE4B1 (pRPDE89 (SEQ ID NO: 5); GenBank™ AF202732); PDE4B2 (pRPDE18 (SEQ ID NO: 10); GenBank™ L27058); PDE4B3 (pRPDE74 (SEQ ID NO: 11); GenBank™ U95748); PDE4B4 (pRPDE90 and pRPDE92; GenBank™ AF202733).

FIG. 4 shows the nucleotide sequence (SEQ ID NO: 1) encoding PDE4B4. The sequences of two plasmids, pRPDE90 and pRPDE92, have been merged. On the merged sequence, pRPDE92 corresponds to nucleotides 1 to 1936, and pRPDE90 corresponds to nucleotides 253 to 2433. This sequence is available as GenBank™ AF202733.

FIG. 5 shows the nucleotide sequence of pRPDE89 (SEQ ID NO: 5), which encodes PDE4B1. This sequence is available as GenBank™ AF202732.

### DETAILED DESCRIPTION OF THE INVENTION

The present invention provides two novel cAMP-specific phosphodiesterase (PDE4B) isoform cDNAs. These cDNAs encode phosphodiesterases, which function in the regulation of physiological processes by hydrolizing cAMP, an intracellular signaling molecule derived from ATP.

The first cAMP-specific phosphodiesterase isoform cDNA is pRPDE90, a phosphodiesterase isolated from a rat (Rattus norwegenesis; Sprague-Dawley strain) cerebral cortex cDNA library cloned into the Eco RI site of Lambda ZAPII, which was obtained from Stratagene. This cDNA encodes a novel PDE4B isoform named PDE4B4 by the inventors in accordance with convention.

PDE4B4 is a novel PDE4B isoform comprising 659 amino acids, 642 of which are shared with the other “long;” isoforms of PDE4B: PDE4B1 and PDE4B2. The remaining 17 amino acids are found at the extreme amino-terminal end of the protein.

The second cAMP-specific phosphodiesterase isoform cDNA of the instant invention is pRPDE89, a novel rat cDNA. pRPDE89 encodes a protein comprising 736 amino acids. This protein is identical in length and 96% identical in amino acids to the human PDE4B1 isoform (712 of 736 amino acids are identical). Without being bound to any particular theory, it appears that pRPDE89 encodes the rat counterpart of the human PDE4B1 isoform of PDE4B.

The present invention provides isolated and purified nucleic acid molecules comprising nucleotides that encode the amino acid sequences of SEQ ID NOS: 2, 4, and 6. In certain embodiments, these nucleic acid molecules comprise nucleotides 262 to 2238 of SEQ ID NO: 1, nucleotides 1 to 51 of SEQ ID NO: 3, and nucleotides 325 to 2532 of SEQ ID NO: 5, respectively. The present invention also provides nucleic acid molecules that encode amino acid sequences that are greater than 90%, greater than 85%, greater than 80%, greater than 75%, and greater than 70% identical to SEQ ID NO: 4. The present invention also provides such nucleic acid molecules subcloned into plasmids; such nucleic acid molecules subcloned into prokaryotic or eukaryotic expression vectors; and such nucleic acid molecules stably or transiently incorporated into a prokaryotic or eukaryotic host cell.

The present invention also provides isolated and purified proteins comprising the amino acid sequences of SEQ ID NOS: 2 and 6 and peptides comprising the amino acid sequence of SEQ ID NO: 4. The present invention further provides antibodies that specifically recognize peptides comprising the amino acid sequence of SEQ ID NO: 4. Such antibodies may be polyclonal or monoclonal antibodies that are prepared according to methods that are well-known in the art. See, e.g., Harlow & Lane, Antibodies: A Laboratory Manual (1988).

Novel PDE4B isoforms such as those of the instant invention are of importance for several reasons. One reason is that the isoforms of the present invention are expressed in brain—an important potential target of PDE4 inhibitors. Indeed, cDNAs encoding numerous PDE4 isoforms have previously been isolated from brain. See e.g., Bolger et al., Mol. Cell Biol. 13:6558-71 (1993), Huston et al., Biochem J. 328:549-56 (1997), McLaughlin et al., J. Biol. Chem. 268:6470-76 (1993), Bolger et al., Gene. 149:237-44 (1993), Davis et al., Proc. Natl. Acad. Sci. U.S.A. 86:3604-08 (1989), Colicelli et al., Proc. Natl. Acad. Sci. U.S.A. 86:3599-3603 (1989), and Engels et al., FEBS Lett. 358:305-10 (1995). The brain is thus a target for many of the actions of selective PDE4 inhibitors. It is therefore important to determine exactly which PDE4 isoforms are present in the brain.

PDE4 inhibitors have several demonstrated effects in the human brain, several of which are beneficial, and others of which are harmful. Some of the potential beneficial effects of PDE4 inhibitors include a demonstrated anti-depressant action. Fleischhacker et al., Neuropsychobiology, 26:59-64 (1992), Eckmann et al., Current Therapeutic Research, 43:291-95 (1988). PDE4 inhibitors may also augment memory and other central nervous system functions. However, PDE4 inhibitors can cause nausea and trigger other gastrointestinal side effects. At least a portion of these deleterious side effects are likely mediated by the action of these drugs in the brain.

Discovery of additional isoforms of the PDE4B phosphodiesterases would open greater possibilities for developing inhibitors that could be specifically targeted at one or more isoforms. Such targeting would allow a more viable approach for utilizing the beneficial properties of these inhibitors in clinical treatment, while selectively avoiding negative side effects.

As a result, a search for novel PDE4 isoforms was initiated in rat brain. Two previously unknown PDE4 isoforms were subsequently isolated. While not being bound to any one particular theory, one of these appears to be the rat homolog of the human PDE4B 1 isoform, which has been described previously in the art. Bolger et al., Mol. Cell Biol. 13:6558-71 (1993). The second novel isoform, called PDE4B4, has a unique 17 amino acid amino-terminal region which is not present in any other PDE4B isoform. It appears likely that PDE4B4 will be similar to other PDE4 isoforms in that it will be highly specific for cAMP and be inhibited by the prototypical PDE4 inhibitor rolipram.

It has previously been shown that the various PDE4 isoforms have different tissue expression patterns. Huston et al., Biochem J. 328:549-56 (1997). Indeed, it has even been shown that different isoforms encoded by the same gene may vary substantially in their tissue expression. (Bolger et al., Mol. Cell Biol. 13:6558-71 (1993), Bolger et al., J. Bio. Chem. 271:1065-71 (1996), and Bolger et al., Gene. 149:237-44 (1994). Studies are in progress to determine the pattern of expression of the four known rat PDE4B isoforms, with special emphasis on their expression in various regions of the brain. Such discoveries and studies create the possibility of exploiting differences in the patterns of tissue expression of the various PDE4 isoforms to “target” the effects of PDE4 inhibitors to specific regions of the brain, thus maximizing their positive effects and minimizing or negating their negative effects.

One current explanation for the divergence of the PDE4B1, PDE4B3 and PDE4B4 mRNAs is alternative mRNA splicing. This has been documented as accounting for the existence of the PDE4A and PDE4D isoforms. Bolger et al., J. Biol. Chem. 271:1065-71 (1996), Bolger et al., Biochem J. 328:539-48 (1997), and Houslay et al., Advances in Pharmacology 44:225-342 (1998). Consistent with this explanation, it has been shown that the point of divergence between PDE4B1, PDE4B3 and PDE4B4 corresponds with the major point of alternative mRNA splicing in the D. melanogaster dunce gene transcripts. It also corresponds with the major point of alternative mRNA splicing in alternatively spliced mRNAs from the human PDE4A (Bolger et al., Mol. Cell Biol. 13:6558-71 (1993)), PDE4B (Huston et al., Biochem J. 328:549-56 (1997)) and PDE4D (Bolger et al., Biochem J. 328:539-48 (1997)) genes. It also corresponds to the 5′ end of an exon in the human PDE4A (Sullivan et al., Biochem J. 333:693-703 (1998)) and murine Pde4a (Olsen & Bolger, Mammalian Genome 11:41-45 (2000)) genes.

In addition, since there is no common 5′ region of sequence at the 5′ ends of any of these cDNAs, it appears likely that each is generated from a different transcriptional start site. It has been previously demonstrated that several murine Pde4a transcripts, including PDE4A5, are generated in this manner (Olsen & Bolger, Mammalian Genome 11:41-45 (2000)).

All references, publications, patents, patent applications, and commercial materials cited in this application are hereby incorporated by reference in their entirety.

### EXAMPLES

The following example is given to illustrate an embodiment which has been made within the scope of the present invention. It is to be understood that the following example is neither comprehensive nor exhaustive of the many types of embodiments which can be prepared in accordance with the present invention.

### Example 1—Two Novel PDE4B Isoforms

Experimental Techniques:

Materials: A rat (Rattus norwegenesis; Sprague-Dawley strain) cerebral cortex cDNA library, cloned into the Eco RI site of Lambda ZAPII, was obtained from Stratagene. All molecular biology, biochemistry and cell culture reagents were from New England Biolabs, Life Technologies or Roche Molecular Systems unless specified otherwise.

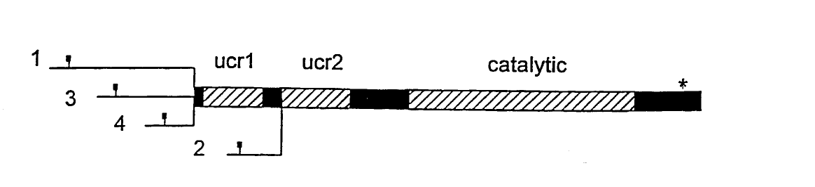

Isolation and Analysis of cDNA Clones: Procedures were as described by Sambrook et al. (Sambrook et al., Molecular Cloning: A Laboratory Manual, (1989)) unless otherwise specified. The cDNA library was screened with a probe corresponding to nucleotides 204 to 1299 of rat PDE4B3 (pRPDE74 (SEQ ID NO: 9) GenBank™ accession number U95748; (Huston et al., Biochem J. 328:549-56 (1997)). This region encodes the unique amino-terminal region of PDE4B3 as well as UCR1 and the majority of UCR2 (FIG. 1). Hybridization was performed with a final wash in 0.3×SSC, 0.3% SDS at 62° C. Sequencing was performed on both strands with an ABI Prism sequencer (Perkin-Elmer) according to the manufacturer's instructions. Alignments were generated with the Gap and Lineup programs of the Wisconsin Package of UNIX sequence software programs (Oxford Molecular Group).

Results:

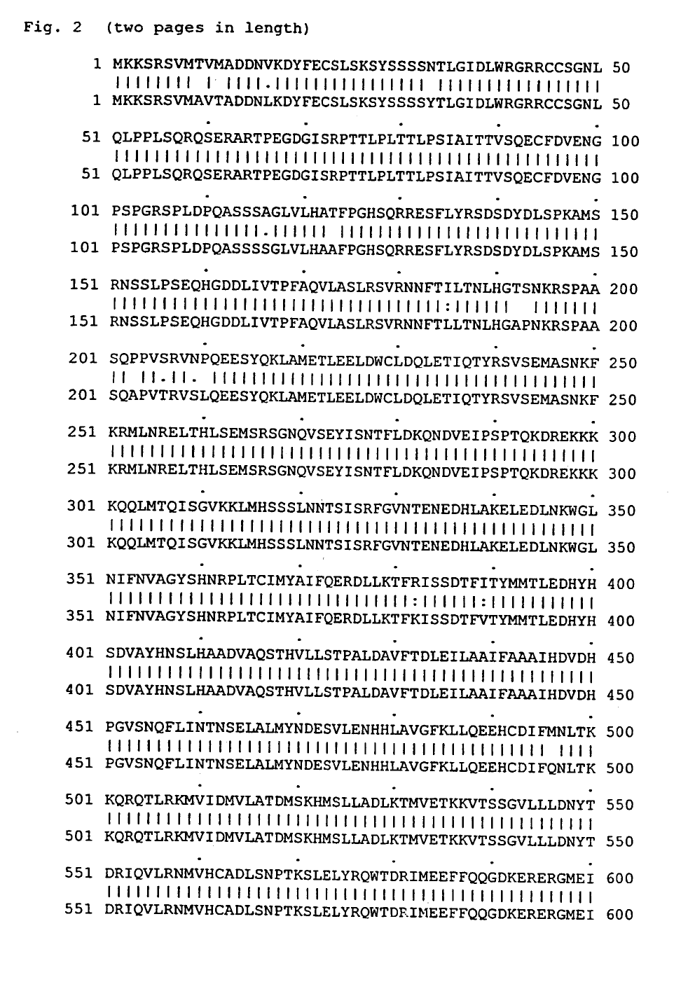

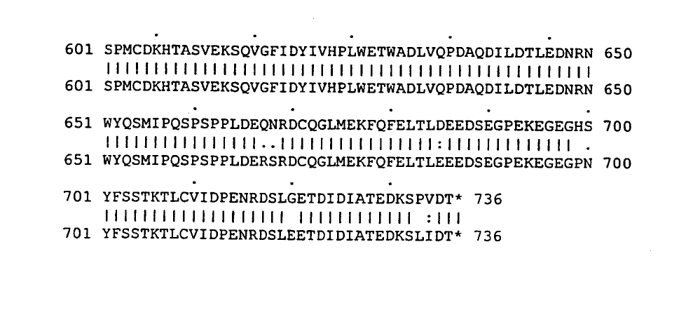

To obtain cDNAs encoding PDE4B isoforms, a rat cortex cDNA library was screened with a probe corresponding to UCR1 and UCR2 of rat PDE4B3 (Huston et al., Biochem. J. 328:549-56 (1997)). This probe was designed to detect all “long” (i.e., UCR1-containing) PDE4B isoforms. cDNAs encoding two different PDE4B isoforms were detected in the screen (see FIG. 1). One cDNA clone, called pRPDE89 (SEQ ID NO: 5), encoded a protein of 736 amino acids (SEQ ID NO: 6). This isoform was identical in length and had greater than 96% amino acid identity (712/736 amino acids identical, FIG. 2) with the human PDE4B1 isoform (SEQ ID NO: 7). Bolger et al., Mol. Cell Biol. 13:6558-71 (1993). It was therefore concluded that pRPDE89 encodes the rat PDE4B1 isoform.

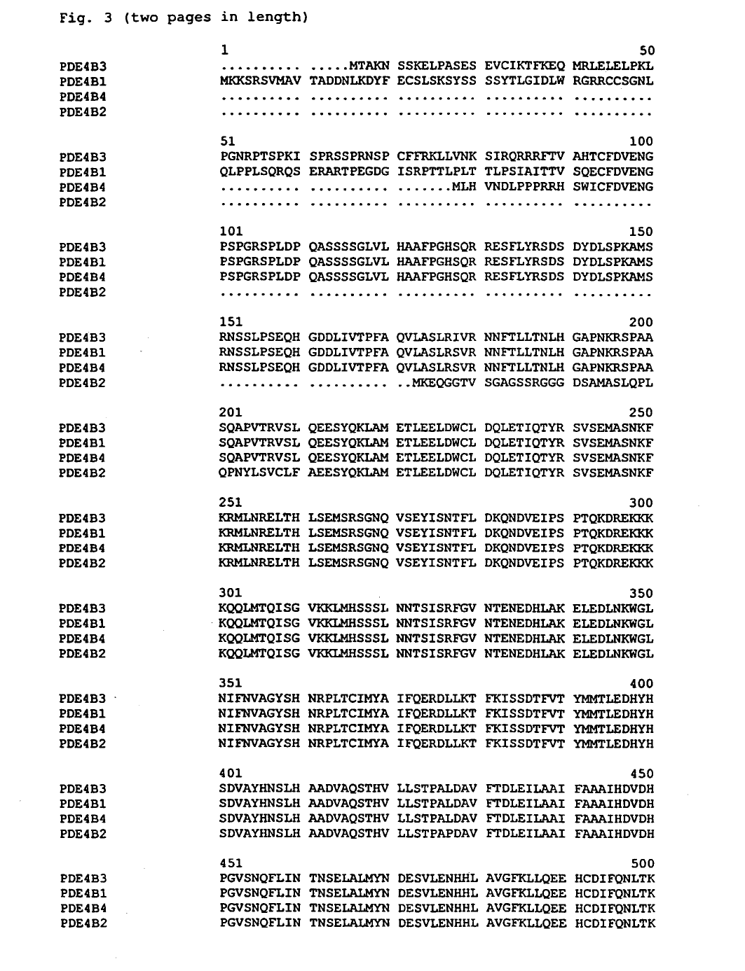

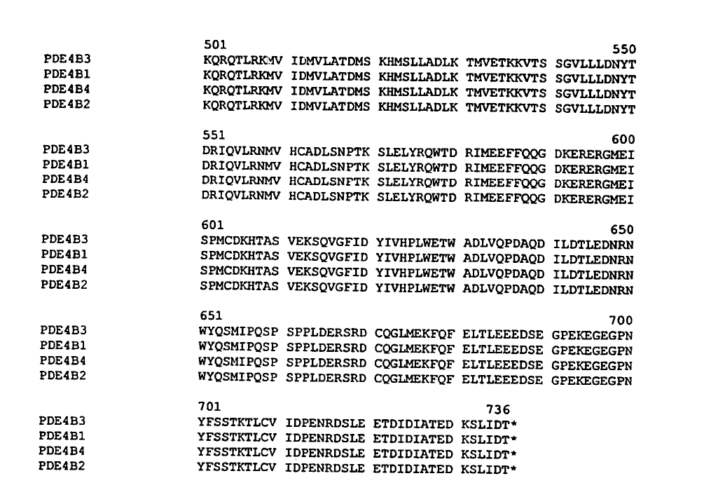

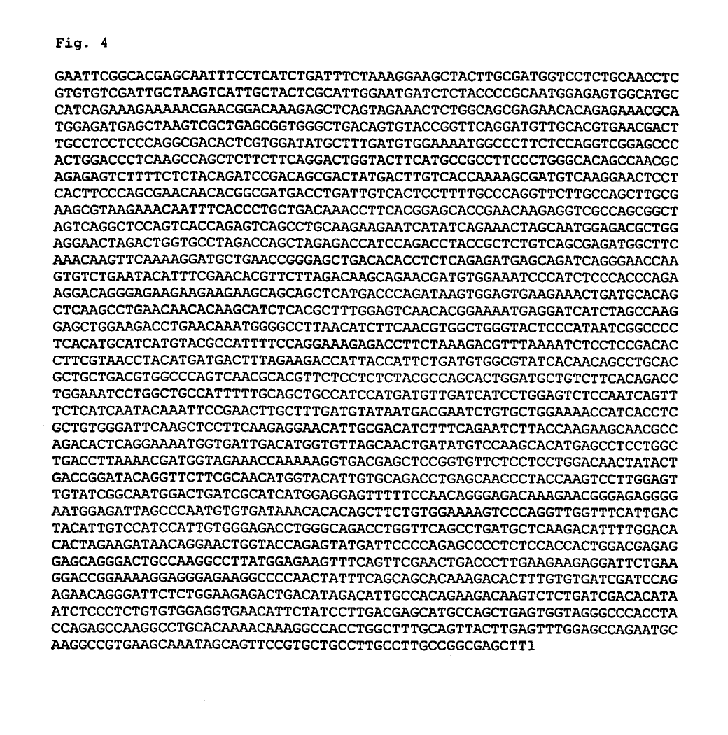

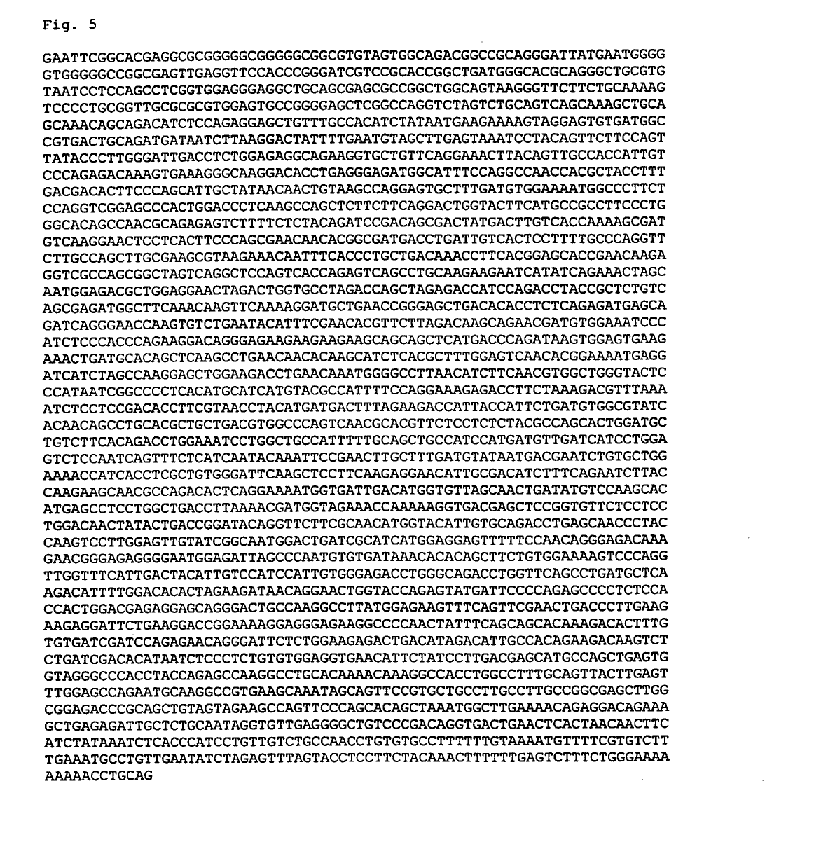

Also detected in the screen was a cDNA clone, called pRPDE90 (SEQ ID NO: 1), which encoded the complete open reading frame of a novel PDE4B isoform. This new isoform was called PDE4B4, using the accepted nomenclature. Beavo, Physiol. Rev. 75:725-48 (1995). The PDE4B4 protein consists of 659 amino acids (SEQ ID NO: 2), 17 of which are located at the extreme amino-terminal end of the protein and show no detectable homology to any previously cloned PDE4B sequence (SEQ ID NOS: 3, 4). The remaining 642 amino acids are identical to the corresponding regions of the “long” PDE4B isoforms PDE4B1 and PDE4B3 (FIG. 3). The nucleotide sequences of the common regions of PDE4B1, PDE4B3 and PDE4B4 are also identical. The sequence of the novel region of PDE4B4 was confirmed by the sequence of another clone isolated in the screen, called pRPDE92 (SEQ ID NO: 10), which completely overlapped the novel region of pRPDE90 and contained sequence of an additional portion of the 5′ untranslated region of the mRNA.

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. An isolated and purified nucleic acid molecule comprising nucleotides encoding the amino acid sequence of SEQ ID NO: 2.

2. The nucleic acid molecule of claim 1, comprising nucleotides 262 to 2238 of SEQ ID NO: 1.

3. The nucleic acid molecule of claim 1, wherein said nucleic acid molecule is subcloned into a plasmid.

4. The nucleic acid molecule of claim 1, wherein said nucleic acid molecule is subcloned into a prokaryotic or eukaryotic expression vector.

5. The nucleic acid molecule of claim 1, wherein said nucleic acid molecule is stably or transiently incorporated into a prokaryotic or eukaryotic host cell.

6. An isolated and purified protein comprising the amino acid sequence of SEQ ID NO: 2.

7. An isolated and purified nucleic acid molecule comprising nucleotides which code for the amino acid sequence of SEQ ID NO: 4.

8. The nucleic acid molecule of claim 7, comprising the nucleotide sequence of SEQ ID NO: 3.

9. An isolated and purified nucleic acid molecule comprising a nucleotide sequence that encodes an amino acid sequence that is at least 90% identical to the amino acid sequence of SEQ ID NO: 4.

10. An isolated and purified nucleic acid molecule comprising a nucleotide sequence that encodes an amino acid sequence that is at least 75% identical to the amino acid sequence of SEQ ID NO: 4.

11. An isolated and purified peptide comprising the amino acid sequence of SEQ ID NO: 4.

12. An antibody that specifically recognizes the peptide of claim 11.

13. An isolated and purified nucleic acid molecule comprising nucleotides encoding the amino acid sequence of SEQ ID NO: 6.

14. The nucleic acid molecule of claim 13, comprising nucleotide 325 to 2532 of SEQ ID NO: 5.

15. The nucleic acid molecule of claim 13, wherein said nucleic acid molecule is subcloned into a plasmid.

16. The nucleic acid molecule of claim 13, wherein said nucleic acid molecule is subcloned into a prokaryotic or eukaryotic expression vector.

17. The nucleic acid molecule of claim 14, wherein said nucleic acid molecule is stably or transiently incorporated into a prokaryotic or eukaryotic host cell.

18. An isolated and purified protein comprising the amino acid sequence of SEQ ID NO: 6.
